(19) United States
(12) Patent Application Publication
(10) Pub. No.: US 2015/0190399 A1
(43) Pub. Date: Jul. 9, 2015

(54) NEW DIFFERENTIAL-RELEASE PHARMACEUTICAL COMPOSITION CONTAINING THREE ACTIVE PRINCIPLES

(71) Applicant: LANDSTEINER SCIENTIFIC S.A. DE C.V., Distrito Federal (MX)

(72) Inventors: Dario Agapito Peralta, Toluca (MX); Julián Martínez Reyes, Nezahualcoyoti (MX); Carlos Alberto Reyes Moreno, Toluca (MX)

(73) Assignee: LANDSTEINER SCIENTIFIC S.A. DE C.V., Distrito Federal (MX)

(21) Appl. No.: 14/416,475

(22) PCT Filed: Jul. 22, 2013
(86) PCT No.: PCT/MX2013/000092

§ 371 Date: Jan. 22, 2015

(30) Foreign Application Priority Data
Jul. 23, 2012 (MX) MX/A/2012008541

(51) Int. Cl. A61K 31/549; A61K 31/4178; A61K 31/435

(52) U.S. Cl. CPC A61K 31/549; A61K 31/435; A61K 31/4178

(57) ABSTRACT

This invention relates to a differential release formulation containing an angiotensin-II receptor antagonist, a calcium channel blocker and a diuretic, wherein the said pharmaceutical composition allows the sequential administration of the active principles in a single dose based on the treatment needs.

### FIELD OF THE INVENTION

The present invention belongs to the field of controlled and differentiated-release pharmaceutical compositions of drugs useful in the treatment of heart conditions.

### BACKGROUND OF THE INVENTION

Drug combinations present certain advantages with respect to the possibility that they can be administered concomitantly in a single pharmaceutical form, thus abating the individual administration of each drug involved.

Several propositions have been developed today that make use of the drug combination to control a condition such as AH, specifically the use of losartan with amlodipine, as described in MULTILAYER PHARMACEUTICAL COMPOSITION COMPRISING TELMISARTAN AND AMLODIPINE (WO/2012/055941); COMBINED PREPARATION FOR THE TREATMENT OF CARDIOVASCULAR DISEASES BASED ON CHRONOTHERAPY THEORY (MXMX/a/2009/003489); PHARMACEUTICAL COMPOSITION COMPRISING AMLODIPINE AND LOSARTAN (MXMX/a/2008/016099).

However, the above compositions speak directly of the combination of losartan with amlodipine, and not of the incorporation of hydrochlorothiazide as renal protector.

Losartan, or losartan potassium, is an angiotensin-II receptor antagonist, indicated in the treatment of: arterial hypertension; heart failure, in combination with diuretics or digitalis (although the latter are increasingly less used in the treatment of heart failure); kidney disease in patients with type 2 diabetes with proteinuria and hypertension, as part of the antihypertensive treatment.

Amlodipine (or amlodipine manufactured by STADA) is a drug belonging to the group of dihydropyridines (see dihydropyridine), which acts as a long-action calcium channel blocker used in medicine as an antihypertensive and in the treatment of angina pectoris. Like other calcium blockers, amlodipine acts by relaxing the smooth muscle of the arterial wall, thus reducing peripheral resistance and, therefore, reducing arterial pressure. In angina pectoris its actions reduce the flow of blood to the heart muscle.

Amlodipine has been indicated for the treatment of arterial hypertension and in the prophylaxis of angina pectoris.

Amlodipine can be used in patients with heart failure; no increase in gross mortality rate has been noted in these cases with the use of amlodipine.¹ Amlodipine tends to improve left ventricular mass, although it is generally used in therapies that combine antihypertensives when the first-line drugs no longer have the desired effect on arterial tension.²

The collateral effects of amlodipine include:

Frequently: Peripheral edema, muscle, stomach and head pain, dyspepsia, nausea in around 1 in 100 users.

Sometimes: Vertigo, palpitations, mammary development in men (gynecomastia), erectile dysfunction, depression, insomnia and tachycardia in 1 in 1,000 users.

Rarely: Erratic behavior, hepatitis, jaundice in 1 in 10,000 users.

Very rarely: Hyperglycemia, tremor in 1 in 100,000 users.

Hydrochlorothiazide, sometimes abbreviated to HCT, HCTZ or HZT is a diuretic drug of first choice belonging to the thiazide group. It acts by inhibiting the capacity of the kidney to retain water, thereby increasing the amount of urine. This reduces the volume of blood, decreasing its return to the heart and thus cardiac output. In addition, through other mechanisms, it is thought to reduce peripheral vascular resistance. Hydrochlorothiazide is sold as a generic medicine and has several commercial names. The appropriate dose of hydrochlorothiazide may vary from patient to patient. It is generally used in arterial hypertension; congestive heart failure; edemas caused by heart, kidney or liver failure; renal diabetes insipidus; renal tubular acidosis; prevention of kidney stones and in idiopathic hypercalciuria.

Based on the above, having two useful drugs in the treatment of arterial hypertension with different action mechanisms (that is, while losartan potassium acts as an angiotensin-II receptor antagonist, amlodipine is a calcium channel blocker) is important because the two drugs do not compete for the same receptors or sites of action.

Calcium channel antagonists like amlodipine are powerful vasodilators that induce a reflex activation both in the nervous system and the RAAS, meaning that there is an increase in angiotensin-II values (by reducing the response to it by blocking the calcium influx required for its effects), as well as a negative sodium balance which enhances the antihypertensive effect of angiotensin-II receptor antagonists. In addition to not having a mechanism of action with common target receptors, the effect would be reflected in their therapeutic action, that is, the hypotensive effect. Furthermore, edema often presents during the administration of amlodipine; in this case the diuretics play a role protecting kidney function.

Clearly, having a medication that provides two or more useful drugs in the treatment of arterial hypertension with different mechanisms of action—losartan potassium and amlodipine with a synergistic effect, plus a diuretic to avoid problems associated with amlodipine—whether by sequential or differential release, would be highly advantageous compared to single-drug medications where the administration of each of the active principles is done separately and in one or two time intervals. In addition, a single dose is required to reach the therapeutic effect of the three drugs, which would guarantee adherence to the treatment.

A drug currently exists that combines losartan with hydrochlorothiazide; however, patent documents have not been published that describe amlodipine-losartan-hydrochlorothiazide combinations.

The Mexican application serial number 2008/16532 describes an amlodipine-hydrochlorothiazide combination; while Mexican patent 198140 discloses a composition containing losartan-hydrochlorothiazide.

MX/a/2011/006008 SOLID PHARMACEUTICAL COMPOSITION COMPRISING AMLODIPINE AND LOSARTAN describes a solid pharmaceutical composition for preventing or treating cardiovascular disorders comprising amlodipine and losartan as active ingredients, and a disintegrant which is a mixture of at least two components selected from the group consisting of sodium starch glycolate, crosscarmellose sodium, and crosspovidone, which exhibits a high and stable level of amlodipine and losartan dissolution rates. MX/a/2011/006009 SOLID PHARMACEUTICAL COMPOSITION COMPRISING AMLODIPINE AND LOSARTAN AND PROCESS FOR PRODUCING SAME refers to a pharmaceutical composition for preventing or treating cardiovascular disorders comprising amlodipine or an acceptable pharmaceutical salt thereof, which exhibits high amlodipine and losartan dissolution rates even under low pH conditions, and improved storage stability. MX/a/2011/006061 SOLID PHARMACEUTICAL COMPOSITION COMPRISING AMLODIPINE AND LOSARTAN WITH IMPROVED STABILITY reveals a solid pharmaceutical composition for preventing or treating cardiovascular diseases comprising granular forms of amlodipine and losartan which are separated from each other, and a stabilizing agent, which has improved storage stability due to minimized interaction between the amlodipine and losartan. And MX/a/2008/016532 SOLID DOSAGE FORMS OF VALSARTAN, AMLODIPINE AND HYDROCHLOROTHIAZIDE AND METHOD OF MAKING THE SAME describes the preparation of monolayer, bilayer and trilayer solid dosage forms of a combination of valsartan, amlodipine and hydrochlorothiazide.

As can be seen, the aforementioned documents do not anticipate in any way the composition subject of this application and which is described below.

### BRIEF DESCRIPTION OF THE DRAWINGS

FIG. 1 shows the dissolution profile of the composition subject of this invention.

### SUBJECT OF THE INVENTION

The aim of this invention is to offer a medication that requires a single dose to achieve the therapeutic effect of three drugs.

A second objective is to provide a multiparticulate product that can be placed inside a capsule or sachet.

A third objective is to offer the patient the comfort of a single dose of the medication once a day, with the advantage that, due to the sequential release of hydrochlorothiazide, the said dose can be taken before going to bed so that the diuretic takes effect the following morning.

An additional aim of this invention is to provide a manufacturing process for a product that combines three active principles, at least one with differential release.

A further objective of this invention is to provide a medication that gives the patient a sense of security and confidence by the immediate treatment of hypertension on the one hand, and the reduction of possible side effects (edemas), typical of amlodipine, on the other.

### SPECIFICATION OF THE INVENTION

Losartan potassium as such possesses a very brief half-life of approximately 2 hours; however, it has an active metabolite that is around 10 to 40 times more powerful whose half-life is between 6 and 9 hours, sufficient time to allow the action of the diuretic drug without any interference between the two. Furthermore, the scheduling of the therapeutic effect is tied to the drug absorption site, hence the effect between the active principles is related, enhancing the therapeutic effect.

The duration of administration of the differentiated or sequential drugs of the combination of the invention is achieved through a solid pharmaceutical form of sustained and sequential release of each drug.

The administration of the composition of this invention in triple-drug pellet form is controlled by an enteric barrier which enables the sequential release of losartan and amlodipine, and the later release of hydrochlorothiazide, while in the case of two-drug tablets, no.

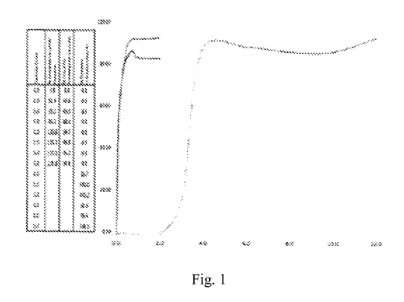

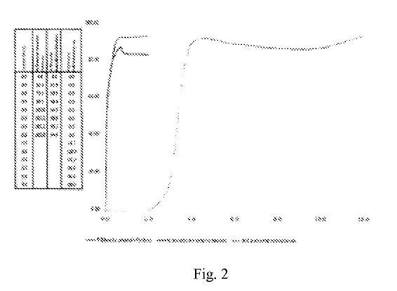

The sequential release of losartan and amlodipine enables the hypertensive effect of one on the other to be synergistic; in addition, the combination contains a diuretic, hydrochlorothiazide, which increases the formation of sodium reabsorption channels which steadily improves the hypotensive effect due to this drug being administered around 4 hours (based on the chart in FIG. 1) after the administration of the medication. This effect has a triple function: first, to increase the hypotensive effect; second, to avoid peripheral edemas that may result from the administration of amlodipine, and third, to encourage the biotransformation of the losartan and amlodipine avoiding the appearance of side effects of the two drugs. Moreover, if this medication is given during the night, the diuretic effect of the hydrochlorothiazide would present in the morning.

Support for the above statements can be found in Fogari R, Zoppi A, Derosa G, Mugellini A, Lazzari P, Rinaldi A, et al. Effect of valsartan addition to amlodipine on ankle oedema and subcutaneous tissue pressure in hypertensive patients. J Hum Hypertens. 2007; 21:220-4; Messerli F H. Vasodilatory edema: a common side effect of antihypertensive therapy. Curr Cardiol Reports. 2002; 4:479-82; and Calhoun D A, Lacourciere T, Chiang Y T, Glazer R D. Triple antihypertensive therapy with amlodipine, valsartan and hydrochlorothiazide: a randomized clinical trial.

The composition subject of this invention comprises hydrochlorothiazide, amlodipine besylate, losartan potassium, a carrier or substrate, a composition of immediate-release water-soluble coating, an extended release, water-soluble composition, and a water-soluble total coating composition. The said composition is prepared in the form of pellets or microspheres packaged in a hard gelatin capsule.

The composition of the immediate release coating is water soluble, regardless of the pH, and allows immediate disintegration and rapid release of the active principle. The said composition comprises, expressed as a % of the total weight of the composition, approximately 52% to 55% polyvinyl alcohol; approximately 305 [sic] talc; approximately between 14% and 16% polyethylene glycol, and approximately 3% polysorbate 80.

The extended release composition forms a film and has plasticizer and stabilizer properties. The composition comprises a primary composition consisting of, expressed as % of total weight of the composition, approximately 60% to 62% ethyl cellulose, approximately 17% oleic acid, approximately 14% to 16% ammonium hydroxide and approximately 6% to 7% hydroxypropyl cellulose; and a secondary composition that is the immediate release composition, at a 9:1 weight ratio.

The composition of total coating, expressed as % of the total weight of the composition, is approximately 40% polyvinyl alcohol, approximately 20% to 22% polyethylene glycol, approximately 18% to 20% titanium dioxide, approximately 14% to 16% talc, and approximately 5% to 7% of a pharmaceutically acceptable pigment.

The coatings described above can be obtained from various suppliers under commercial brands including, Opadry II, Opadry II Clear, Opadry II with pigment, Surelease Clear, and the like.

The new differential release pharmaceutical composition subject of this invention comprises approximately 5% to 20% in weight of the composition of losartan potassium, approximately 0.5% to 3% in weight of amlodipine, from 1% to 4% in weight of hydrochlorothiazide, approximately 55% to 65% in weight of the cellulose sphere or sugar starch type carrier or substrate, approximately 2% to 4% of a primary water-soluble immediate release coating, approximately 7% to 9% of a second water-soluble extended release coating, approximately 2% to 4% of a third water-soluble immediate release coating, approximately 4% to 6% of a fourth water-soluble immediate release coating and approximately 6% to 8% of a layer of immediate dissolution water-soluble total coating.

The composition of this invention has the form of pellets or microspheres, which are poured into a hard gelatin capsule for distribution.

An example of the composition of this invention is shown in Table 1.

TABLE 1

| Component | mg |
|---|---|
| Sugar-starch spheres | 308.8 |
| Hydrochlorothiazide | 12.5 |
| Opadry II Clear 85F 19250* | 11.3 |
| Surelease Clear E-7-19050** | 36.7 |
| Opadry II Clear 85F 19250* | |
| Amlodipine besylate | 6.9 |
| Opadry II Clear 85F 19250* | 25 |
| Losartan potassium | 50 |
| Opadry II Clear 85F 19250* | |
| Opadry II Blue 85F 90631** | 40 |

*Contains 52.6% polyvinyl alcohol; 30% talc; 14.74% polyethylene glycol and 3% polysorbate.
**Contains 61.23% ethylene cellulose; 17% oleic acid; 14.96% ammonium hydroxide and 6.8% hydroxypropyl cellulose.
***Contains 66.66% polyvinyl alcohol; 30% talc; 33% polyethylene glycol and 3% polysorbate.

The composition in Table 1 is manufactured based on the following process:

The sugar-starch spheres are loaded into a fluid bed apparatus with the necessary fixtures to perform the coating of the microspheres. It is heated for at least 15 minutes to a temperature between 60° C. and 70° C.

The first coating dispersion is prepared separately placing 960 g of purified water in a container and adding Opadry II Clear 85F19250 under agitation. Once the mixture is uniform, hydrochlorothiazide is added, continuing agitation until fully incorporated.

The above dispersion is added over the microsphere substrate, maintaining the inlet temperature at between 45° C. and 55° C.

The second coating dispersion is prepared separately placing 760 g of purified water in a container and then adding Opadry II Clear 85F19250. Once incorporated, Surelease Clear E-7-19250 is added.

The above mixture is added to the substrate and first coating mixture maintaining the inlet temperature at between 55° C. and 65° C.

A third coating dispersion is prepared placing 680 g of purified water in a container and incorporating Opadry II Clear 85F19250. Once incorporated, amlodipine besylate is added.

The coating is applied maintaining the inlet temperature at between 50° C. and 60° C.

The fourth coating layer is prepared placing 2400 g of purified water in an appropriate container under constant agitation and incorporating Opadry II Clear 85F19250. Once incorporated, 400 g of losartan potassium is added.

The coating is applied maintaining the temperature at between 50° C. and 60° C.

The fifth coating layer is prepared placing 1600 g of purified water in an appropriate container under constant agitation and adding Opadry II Blue 85F90631 until completely uniform.

The coating is applied maintaining the temperature at between 50° C. and 60° C.

Once the impregnation process is finished, the pellets are unloaded from the bowl. 500 mg of pellets is filled into Size 0 natural/natural capsules.

The composition obtained from Table 1 shows a dissolution profile represented in FIG. 1, where it can be seen that losartan and amlodipine are released immediately, as required in the treatment of hypertension, and hydrochlorothiazide is released up to six hours later, which is when it imparts its therapeutic effect aimed at reducing symptoms of edema in the patient.

These compositions are given by way of example, but in no way limit the scope of the description of this invention.

1. A new differential release pharmaceutical composition characterized in that it comprises hydrochlorothiazide, amlodipine besylate, losartan potassium, a carrier or substrate, an immediate release water-soluble coating composition, an extended release water-soluble coating composition and a water-soluble total coating composition.

2. The composition according to claim 1, characterized in that the immediate release coating is water soluble, regardless of the pH and allows an immediate disintegration and rapid release of the active principle; the said composition comprising, expressed as % of the total weight of the composition, approximately 52% to 55% polyvinyl alcohol; approximately 305 [sic] talc; approximately between 14% and 16% polyethylene glycol and approximately 3% polysorbate 80.

3. The composition according to claim 1, characterized in that the extended release coating composition forms a film, having plasticizer and stabilizer characteristics.

4. The composition according to claim 3, characterized in that the said coating composition comprises a primary composition comprising, expressed as % of the total weight of the composition, approximately 60% to 62% ethyl cellulose, approximately 17% oleic acid, approximately 14% to 16% ammonium hydroxide and approximately 6% to 7% hydroxypropyl cellulose; and a secondary composition that is the immediate release composition according to claim 2; in addition, the weight ratio of the primary composition to the second is 9:1.

5. The composition according to claim 1, characterized in that the total coating composition, expressed as % of the total weight of the composition, comprises approximately 40% polyvinyl alcohol; approximately 20% to 22% polyethylene glycol; approximately 18% to 20% titanium dioxide; approximately 14% to 16% talc, and approximately 5% to 7% of a pharmaceutically acceptable pigment.

6. The composition according to claim 1, characterized in that it comprises approximately 5% to 20% in weight of the losartan potassium composition; approximately 0.5% to 3% in weight of amlodipine; of 1% to 4% in weight of hydrochlorothiazide; approximately 55% to 65% in weight of the cellulose spheres or sugar starch type carrier or substrate; approximately 2% to 4% of an immediate release water-soluble primary coating; approximately 7% to 9% of an extended release water-soluble secondary coating; approximately 2% to 4% of an immediate release water-soluble tertiary coating; approximately 4% to 6% of a fourth immediate release water-soluble coating, and approximately 6% to 8% of an immediate dissolution water-soluble layer.

7. The composition according to claim 1, characterized in that it has the form of pellets or microspheres which are poured into a hard gelatin capsule for distribution.

8. A process for the manufacture of the composition according to claim 1, the said process characterized in that it comprises the following stages:
A. covering the carrier or substrate of sugar-starch spheres with the first coating dispersion in a fluid bed apparatus at a temperature of between 60° C. and 70° C.;
B. adding hydrochlorothiazide at an inlet temperature of between 45° C. and 55° C. and agitating until fully incorporated;
C. adding the second coating dispersion;
D. mixing at an air inlet temperature of between 55° C. and 65° C.;
E. incorporating the third coating, and once incorporated, adding amlodipine besylate at an inlet temperature of between 50° C. and 60° C.;
F. incorporating the fourth coating under constant agitation, and once incorporated adding losartan potassium at a temperature between 50° C. and 60° C.;
G. adding the fifth coating until completely uniform, at a temperature between 50° C. and 60° C.

9. The process according to claim 8, characterized in that the coatings are prepared separately.

10. The process according to claim 8, characterized in that pellets are obtained, and in that the said pellets are encapsulated in hard gelatin natural/natural capsules.
